(19) United States
(12) Patent Application Publication
(10) Pub. No.: US 2026/0263307 A1
(43) Pub. Date: Sep. 10, 2026

(54) DEVICES TO TREAT AND/OR ENHANCE NAIL GROWTH

(71) Applicant: Christopher B. STEWART, Charlottesville, VA (US)

(72) Inventor: Christopher B. STEWART, Charlottesville, VA (US)

(21) Appl. No.: 19/658,057

(22) Filed: Apr. 24, 2026

Related U.S. Application Data
(63) Continuation-in-part of application No. 19/349,701, filed on Oct. 3, 2025.
(60) Provisional application No. 63/715,697, filed on Nov. 4, 2024.

(51) Int. Cl. A61H 1/02

(52) U.S. Cl. CPC A61H 1/0218; A61H 1/0288

(57) ABSTRACT

Device kits to assist in treatment and/or enhancement of nail growth of a digit for a patient in need thereof are provided with an anchor member adapted to being affixed to a subjacent portion of the nail of a patient’s digit and a flexible traction strip. The traction strip is adapted to being connected to the affixed anchor member so as to extend around the patient’s digit and be connected at an opposite end thereof so as to exert a tension force onto the nail of the patient in a predetermined direction relative to the nail midline to assist in treatment and/or enhancement of nail growth. The anchor strip is preferably a flexible hook-and-loop fastener strip having hooks on one surface thereof and loops on an opposite surface thereof. Symmetrical or asymmetrical tension force may be exerted onto the nail to encourage directional nail growth.

### CROSS REFERENCE TO RELATED APPLICATIONS

This application is a continuation-in-part of U.S. Patent Application Serial No. 19/349,701 filed on Oct. 3, 2025, which in turn is based on and claims domestic priority benefits from U.S. Provisional Application Serial No. 63/715,697 filed on Nov. 4, 2024, the entire content of each application being expressly incorporated hereinto by reference.

### FIELD

The embodiments disclosed herein relate generally to device kits that are especially useful to promote nail health and growth, particularly following trauma or surgical removal of the nail. Specifically, the embodiments of the device kits disclosed herein offer a relatively gentle distraction designed to encourage physiological processes that enhance nail regeneration and directional nail growth.

### BACKGROUND

Following traumatic or surgical nail removal, patients often experience delayed and/or angulated nail growth and discomfort. This period of growth can range from months to greater than one year in some instances. Traditional methods for managing pain and anxiety during recovery include medications and topical treatments, but these do not specifically address the need for physiologic distraction and the encouragement of nail regrowth. Human nails are protective by design. It is well understood that the sooner nails grow back the sooner they can perform their protective function thereby allowing individuals to resume normal activity without pain or hesitation.

Various nail corrective devices are known in the art, e.g., from U.S. Patent Nos. 8,137,297 and 8,262,600, the entire contents of each being expressly incorporated hereinto by reference. While such conventional nail corrective devices may serve their intended purposes, improvements are still desired. It is therefore towards providing such improvements that the embodiments of the present invention disclosed herein are directed.

### SUMMARY

Broadly, the embodiments disclosed herein are directed toward device kits that are especially useful to promote nail health and growth by exerting a directional tension force onto a patient’s nail. The directional tension force may by symmetrical to encourage nail growth in a symmetrical direction coincident to a patient’s digit midline or may be angularly asymmetrical to redirect nail growth which is angularly offset relative to the patient’s digit midline (e.g., so as to treat an ingrown nail or promote asymmetrical nail growth). The device kits disclosed herein may be employed for nail growth enhancement for fingers and/or toes of a patient. The device kits of the embodiments disclosed herein are thereby usefully employed so as to, for example, increase nail growth, correct angular deformities of nails and/or treat ingrown nails by redirecting the direction of nail growth.

According to exemplary embodiments, device kits to assist in treatment and/or enhancement of nail growth of a digit for a patient in need thereof are provided with an anchor member adapted to being affixed to a portion of a nail of the patient’s digit in need of treatment and/or growth enhancement. An anchor strip having a sufficient length is adapted to be wrapped around the digit proximal to the nail and is connectable to one end of a traction strip. The traction strip is of sufficient length so as to be extended distally (forwardly) around a distal end of the patient’s digit and redirected proximally (rearwardly) under the digit to allow an opposite end thereof to be attached to the anchor strip. The traction strip is thereby capable of exerting a predetermined tension to the nail of the patient to assist in treatment and/or enhancement of nail growth by attachment thereof to the anchor member affixed to the nail.

An adhesive material (e.g., a UV-curable resin) may be provided which is adapted to adhesively affix the anchor member to the nail of the patient. At least one (preferably both) of the anchor strip and the traction strip may be a conventional flexible hook-and-loop fastener strip or tape (e.g., VELCRO-Brand fastener) having relatively stiff hooks on one surface and relatively soft fibrous loops on an opposite surface thereof. As is well known, bringing the hooks into contact with the loops will cause the former to releasably interlock with the latter thereby providing a reliable fastening system.

According to some embodiments, the anchor member will include a base element adapted to being adhesively affixed to the nail of the patient and a J-shaped anchor element which is oriented proximally when the anchor member is affixed to the nail of the patient. Alternatively, the anchor member the anchor member is in the form of a substantially flat tab having one end adapted to being adhesively affixed to the nail of the patient and an opposite unaffixed free end. The opposite end of the flat anchor tab member may define an opening so as to allow connection to one end of the traction strip.

The anchor member may alternatively be provided as a substantially flat tab having one end adapted to being affixed to the nail of the patient’s digit and an opposite unaffixed free end. The opposite unaffixed free end of the anchor member in such an embodiment may therefore define an opening adapted to being connected to the traction strip. The one end of the anchor member that is attachable to the nail of the patient’s digit may be provided with an adhesive region which is adapted to adhesively affix the one end of the anchor member to a subjacent portion of the nail. A removable protective release film may optionally be provided to cover such an adhesive region.

According to other embodiments, the anchor member may be a metal post (e.g., formed of magnetic stainless steel). The metal post may therefore include a generally circular base adapted to be directly bonded to the nail, a central pedestal column extending outwardly from the base and an enlarged head at terminal end of the central pedestal column which is spaced from the base. One end of the traction strip will therefore be provided with a flexible thread or wire loop extending outwardly therefrom which is sized and adapted to being positioned over the enlarged head and around the central pedestal of the metal post.

The device kits herein may be provided with a UV-light emitting device for curing the UV-curable adhesive of the kits when applied to the nail to thereby adhesively affix the anchor member to the nail when cured. The UV-light emitting device may comprise a UV-light pen.

According to some embodiments, an attachment wand is provided with the kits which includes a magnetic attachment member at a distal end thereof. The magnetic metal anchor member may thereby be magnetically attached to the anchor member to enable manipulation of the anchor member relative to the applied adhesive site on the patient’s nail. Once applied adhesive has cured, the anchor member may be magnetically separated from the anchor member which remains affixed to the nail. If a UV-curable adhesive is employed, a UV-light can be emitted to effect curing of the adhesive and then subsequent separation of the magnetic attachment member from the anchor member. A UV-light emitting collar may therefore be provided as part of the attachment wand proximal to the magnetic attachment member so as to allow placement of the anchor member and UV-curing of the adhesive with one-handed operation.

In use, the anchor member is affixed to a subjacent portion of the nail while the anchor strip is wrapped around a patient digit proximal to the nail. One end of the traction strip may thereby be connected to the anchor member such that the traction strip extends anteriorly around and over the end of the patient’s digit and is redirected proximally and inferiorly (under) the patient’s digit. An opposite end of the traction strip may then be connected to the anchor strip so that the traction strip exerts a predetermined directional tension force on the nail of the patient through the anchor member affixed thereto.

These and other aspects and advantages of the present invention will become clearer after careful consideration is given to the following detailed description of the preferred exemplary embodiments thereof.

### BRIEF DESCRIPTION OF ACCOMPANYING DRAWINGS

The disclosed embodiments of the present invention will be better and more completely understood by referring to the following detailed description of exemplary non-limiting illustrative embodiments in conjunction with the drawings of which:

FIG. 1 is a plan view of kit components for a device in accordance with an embodiment of the invention which is adapted to treat and/or enhance nail growth of a patient in need of the same;

FIGS. 2-7 show an exemplary sequence of assembling the kit components shown in FIG. 1 onto a nail of a patient to construct the device in accordance with the invention to treat and/or enhance the patient’s nail growth;

FIG. 8 is an enlarged plan view showing an alternative embodiment of an anchor member that may be employed in the kit of FIG. 1;

FIG. 9 is a perspective view showing the manner in which the anchor member of FIG. 8 can be installed during the assembly of the device in accordance with another embodiment of the invention;

FIG. 10 is perspective view of another possible embodiment of kit components of the invention;

FIG. 10A is an enlarged view of the anchor member employed in the kit shown in FIG. 10;

FIGS. 11-18 depict a sequence showing the application of the kit components shown in FIG. 10 onto a nail of a patient in need of nail growth treatment and/or enhancement;

FIGS. 19-21 depict an alternate sequence of adhesively securing the anchor member to a portion of a patient’s nail using an applicator wand provided with a magnetic attachment member and a UV-light emitting collar at a distal end thereof; and

FIG. 22 depicts an exemplary alternative use of the kits described herein to provide a tension force which asymmetrically redirects nail growth relative to the digit midline (e.g., so as to treat an ingrown nail by exerting a substantially transverse force to the nail relative to the dgit midline).

### DETAILED DESCRIPTION OF EXEMPLARY EMBODIMENT

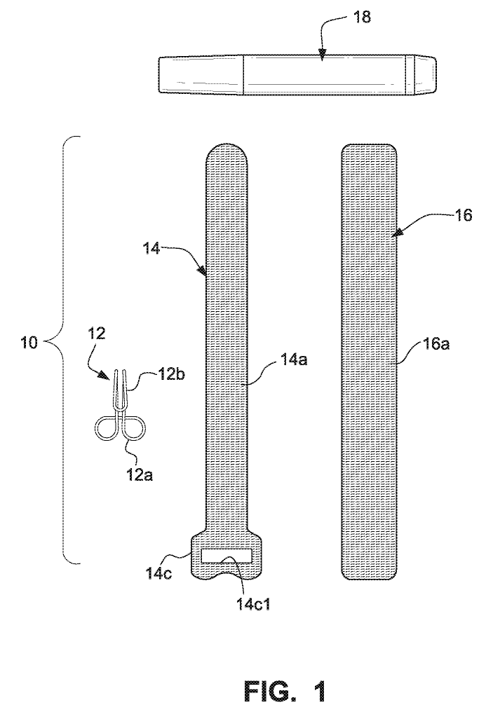

Accompanying FIG. 1 depicts the component parts of a device in kit form 10 which are adapted to being assembled to form a device that assists in the treatment and/or enhancement of nail growth for a patient in need thereof. In essence, the device kit 10 will include an anchor member 12, an elongate traction strip 14 and an elongate anchor strip 16.

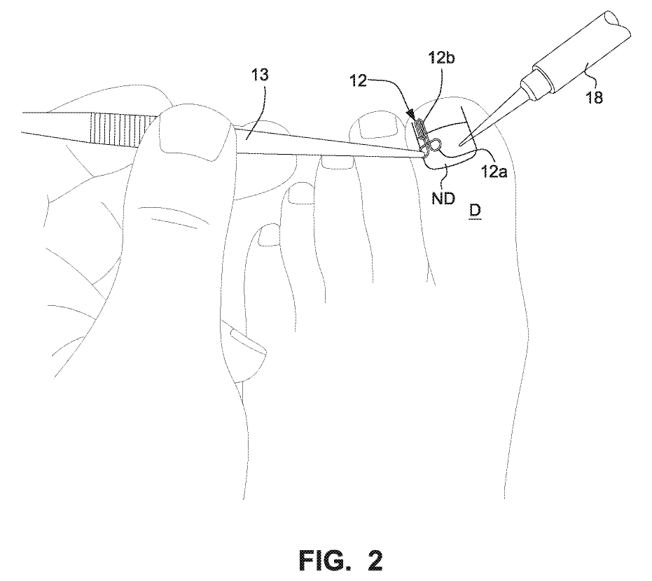

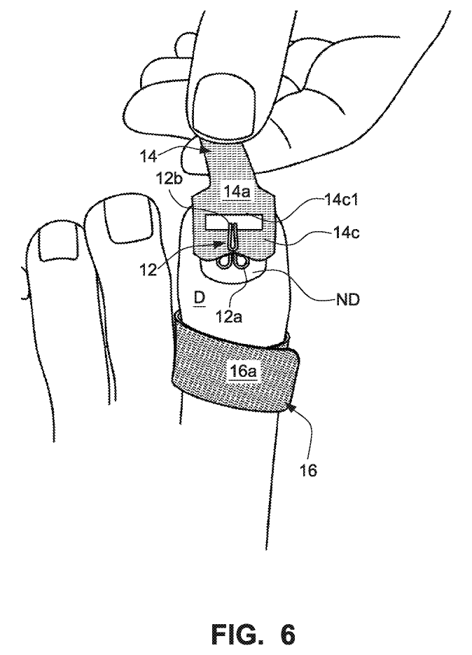

In the embodiment shown in FIGS. 1-7, the anchor member 12 is in the form of a J-shaped component having a base portion 12a and a hook portion 12b. The base portion 12a is adapted to being immovably fixed to a portion of the patient’s nail ND (see FIGS. 2-7) by a suitable nail compatible adhesive. In this regard, the device kit 10 may include a vial 18 containing a suitable adhesive, e.g., a liquid cyanoacrylate adhesive that is conventionally employed to attach synthetic nails to natural nails. When cured, therefore, the adhesive will immovably fix the base portion 12a of the anchor member 12 to a desired subjacent portion of the patient’s nail ND such that the hook portion 12b extends upwardly and proximally relative to the nail ND. The anchor member 12 can be formed of any metal or plastic material that is compatible with the adhesive. In the embodiment depicted, the anchor member is in the form of a metal wire element that is bent into a J-shaped member forming the base and hook members 12a and 12b, respectively.

Each of the elongate traction and anchor strips 14 and 16, respectively, are preferably flexible strips or tapes of hook-and-loop fasteners (e.g., VELCRO-Brand fasteners) respectively having a dense plurality of hooks on one side surface 14a, 16a thereof and a dense plurality of pile loops on an opposite side surface 14b, 16b, thereof. As is well known, a secure yet releasable attachment occurs when a portion of the hooks on one side 14a, 16a of the fastener are brought into engagement with a respectively corresponding portion of the loops on the opposite side 14b, 16b of itself or another fastener.

The traction strip 14 may include a head portion 14c at one end thereof which defines a transverse slot 14c1. The traction strip 14 may thus be in the form of a conventional flexible tie wrap, such as a VELCRO-Brand ONE WRAP® Tie element. The anchor strip 16 can be the same or different from the traction strip 14. Thus, in the embodiment shown, the anchor strip 16 is in the form of an elongate VELCRO-Brand strip that does not include a head portion. However, VELCRO-Brand ONE WRAP® Tie element similar to that employed for the traction strip 14 could also suitably be employed as the anchor strip 16.

An exemplary sequence of component assembly of the device kit 10 is shown in FIGS. 2-7. In this regard, the anchor member 12 is initially attached to a desired surface portion of the patient’s nail ND associated with a patient’s digit D1 (which in the embodiment shown is exemplified by the patient’s large toe but could be any other digit of the patient) by applying a suitable amount of adhesive from the vial 18 and then positioning the base element 12a of the anchor member 12 onto the applied adhesive such that the J-shaped hook element 12b is oriented in a proximal direction. The use of tweezers 13 may be employed to assist in the positioning of the Once the adhesive has cured, the anchor member 12 will thus be immovably fixed to the nail ND.

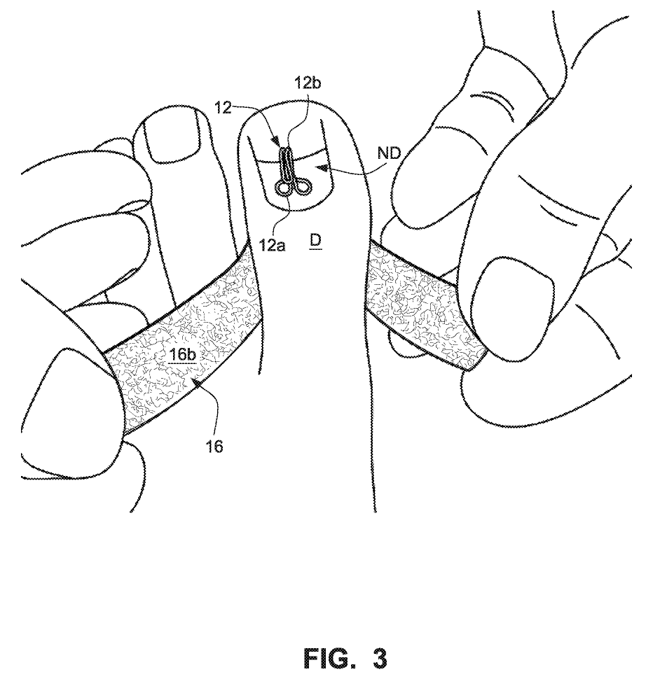

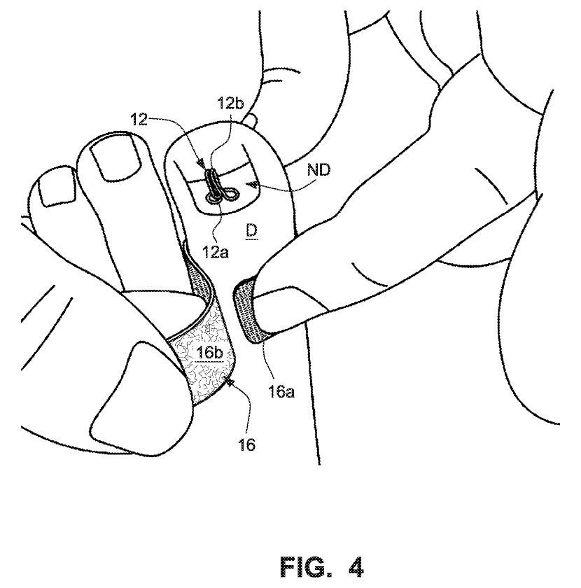

Before, during or after curing of the adhesive which fixes the anchor member 12, the anchor strip 16 may be wrapped around the patient’s digit D proximally (posteriorly) of the nail ND as shown in FIGS. 3-4 and secured to itself as shown in FIG. 4 by bringing the loop side 16b of the strip 16 into engagement with respective portions of the hook side 16a of the strip 16. When wrapped around the patient’s digit D, the loop side 16b of the strip 16 is preferably the bottom side so as to contact the patient’s skin and thereby promote comfort, while the hook side 16a is preferably positioned as the upper side of the strip 16 to thereby engage a portion of the loop side 16b after the strip 16 is wrapped around the digit D.

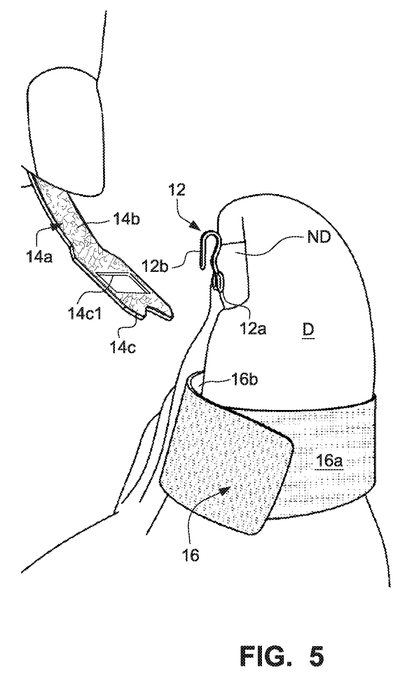

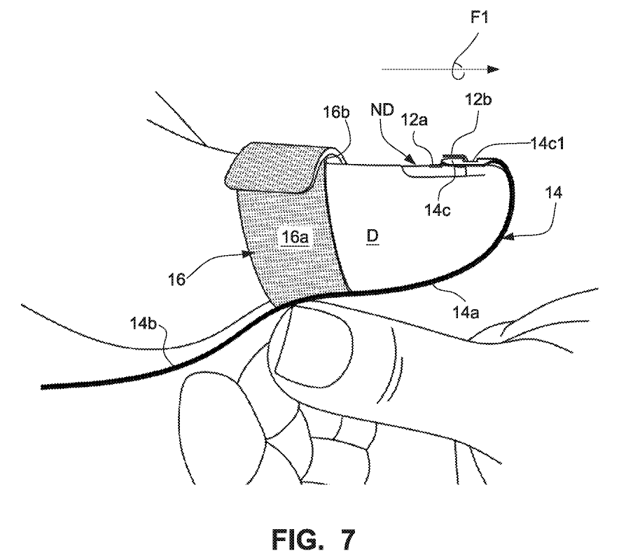

As is shown in FIGS. 5-7, the traction strip 14 may then be positioned relative to the distal (anterior) tip end of the patient’s digit D with the relatively soft loop side 14b preferably facing down so it is against the patient’s skin. The slot 14c1 defined by the head 14c of the traction strip 14 will receive the J-shaped hook member 12b to allow the traction strip 14 to be extended forwardly around the distal (anterior) tip end of the patient’s digit D and then pulled proximally (rearwardly) under the digit D to allow an tail end portion of the traction strip 14 to be engaged with a corresponding portion of the anchor strip (e.g., by bringing the loop side 14b of the end portion of the traction strip 14 into engagement with a corresponding portion of the hook side 16a of the anchor strip 16). It will be appreciated that once secured to the anchor strip 16, the traction strip 14 will exert a desired amount of tension force (arrow F₁ in FIG. 7) in a distal direction relative to the patient’s digit D which is transferred onto the nail ND by the anchor member 12. Any excess length of the traction strip 14 that may extend proximally beyond the anchor strip 16 after being secured may be severed and discarded.

The distal tension force (arrow F₁ in FIG. 7) applied to the nail ND by the traction strip 14 when installed in the manner described above may be symmetrical (e.g., the tension force will be exerted on the nail ND in substantial alignment with the centerline of the digit D) or asymmetrical (e.g., the tension force will be offset relative to the centerline of the digit D). Symmetrical tension force is desired if the nail ND is itself substantially symmetrical and non-angulated. In those situations where the nail ND is asymmetrically angulated, it may be desirable to exert a greater tension force on one side of the nail ND as compared to the other.

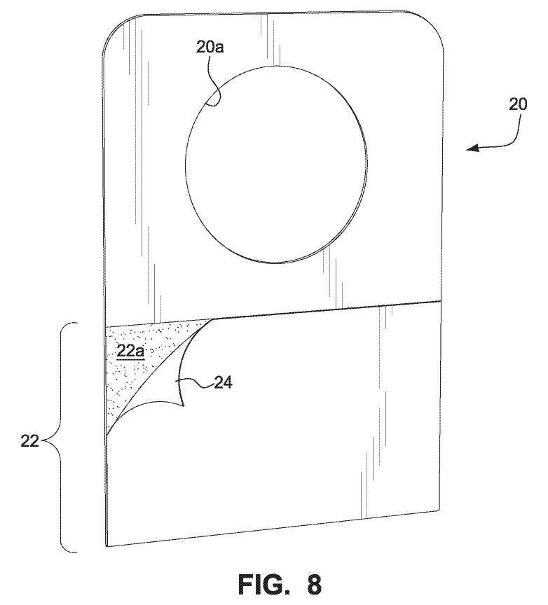

An alternative embodiment of the anchor member 20 to the anchor member 12 discussed previously is shown in FIG. 8. Specifically, the anchor member 20 as shown in the embodiment of FIG. 8 is preferably a flat sheet-like tab formed from plastic or metal material. A lower underside portion of the anchor member 20 may be provided with an adhesive region 22 is provided with a layer of adhesive 22a covered by a protective release film 24 prior to use. The anterior (forward) end portion of the anchor member 20 defines an opening 20a that is sized and configured to receive therein the traction strip 14 as described previously.

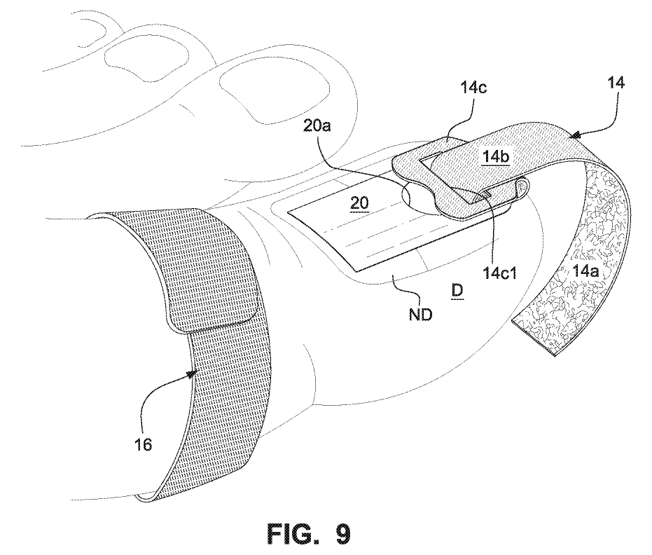

In order to assemble the anchor member 20 to the nail ND, the release film 24 is first removed to expose the adhesive region 22. The adhesive region 22 may thus be brought into contact with a surface portion of the nail ND so that an anterior (forward) portion of the anchor member 20 which defines the opening 20a is positioned near the distal end of the patient’s digit D. The anterior portion of the anchor member 20 is therefore unattached to the nail ND and may be flexed upwardly sufficiently to allow the traction strip 14 to be looped through the opening 20a. Such a state is shown in FIG. 9. Alternatively, the traction strip 14 may be looped through the opening 20a prior to securing the anchor member 20 to the nail ND. Thereafter, the traction strip 14 may be secured to the anchor strip 16 in a manner similar to that shown in FIG. 7 and described hereinabove.

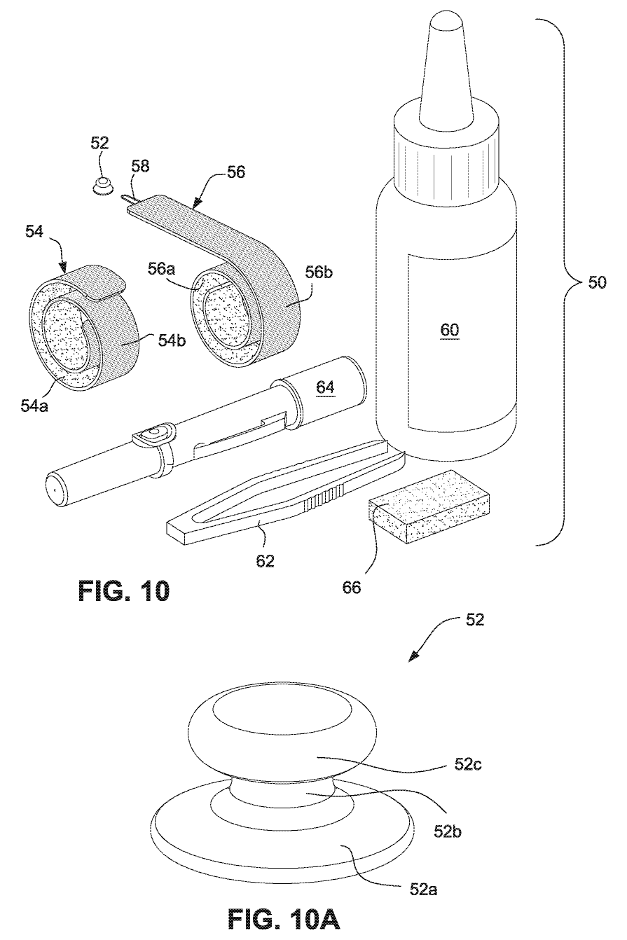

A further embodiment of a kit 50 which embodies the invention is shown schematically in FIG. 10 as including an anchor member 52, an elongate anchor strip 54, and an elongate traction strip 56. The traction strip 56 includes a loop 58 formed of e.g., suture thread or relatively thin flexible wire, that is fixed to a terminal end of the strip 56 so as to extend outwardly therefrom along the strip longitudinal axis. The size of the loop is sufficiently large to be positioned over the anchor member 52 as will be explained in greater detail below. It is especially preferred that the kit 50 also be provided with a supply of UV curable adhesive 60, tweezers 62 and a UV-light emitting light pen 64. A nail buffing pad 66 may also optionally be provided to buff the nail ND in preparation for fixing the anchor member 52 thereto with the UV-curable adhesive 60.

Each of the elongate anchor strip 54 and the elongate traction strip 56 is preferably a conventional hook-and-loop fastener strip or tape (e.g., VELCRO-Brand fastener) having relatively stiff hooks on one surface 54b, 56b thereof and relatively soft fibrous loops on an opposite surface 54a, 56a thereof. The anchor and traction strips 54, 56, respectively, may be provided in predetermined lengths sufficient to accommodate varying sizes of human digits. Any excess length of the anchor and traction strips 54, 56, respectively, may be severed and discarded once proper sizing has been determined for a particular patient.

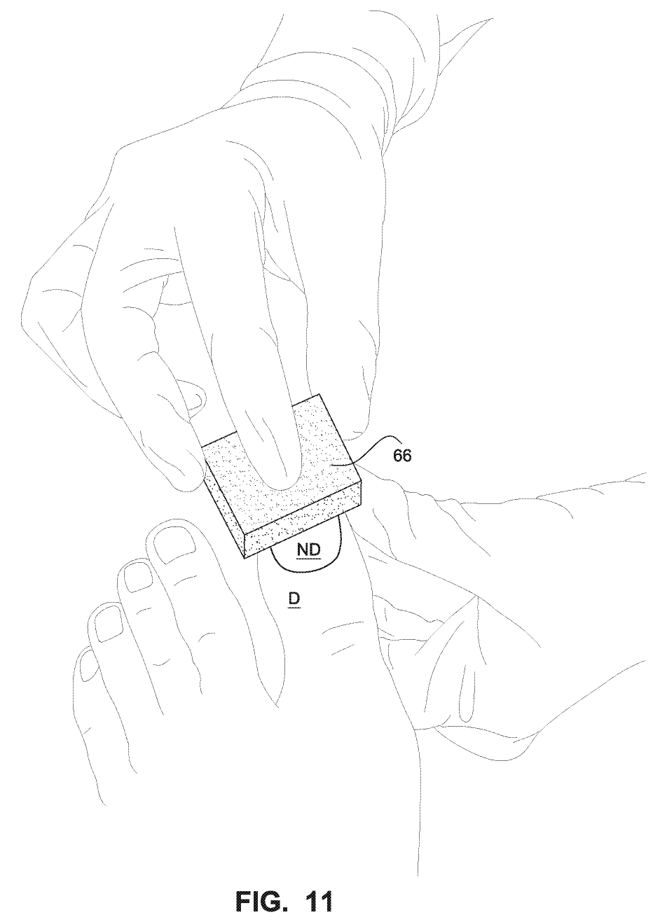

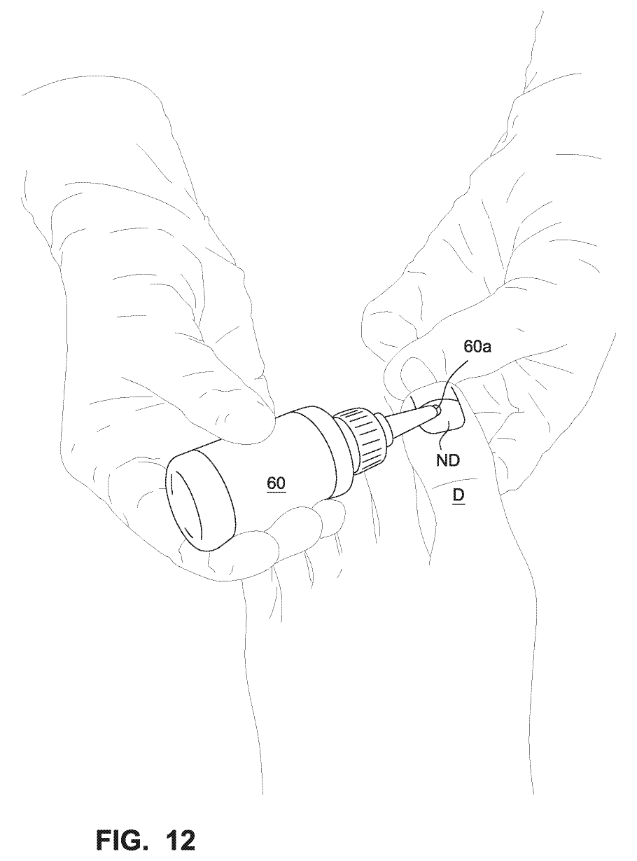

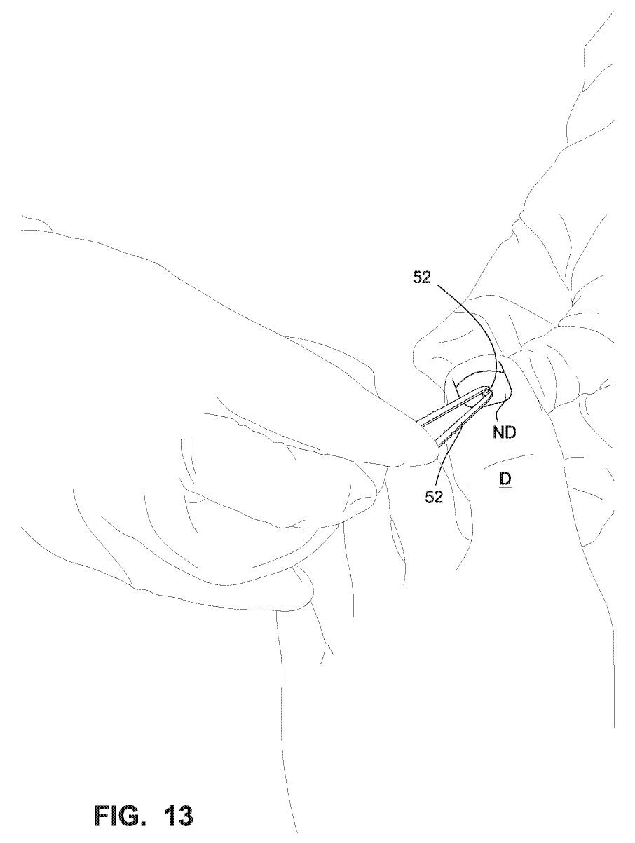

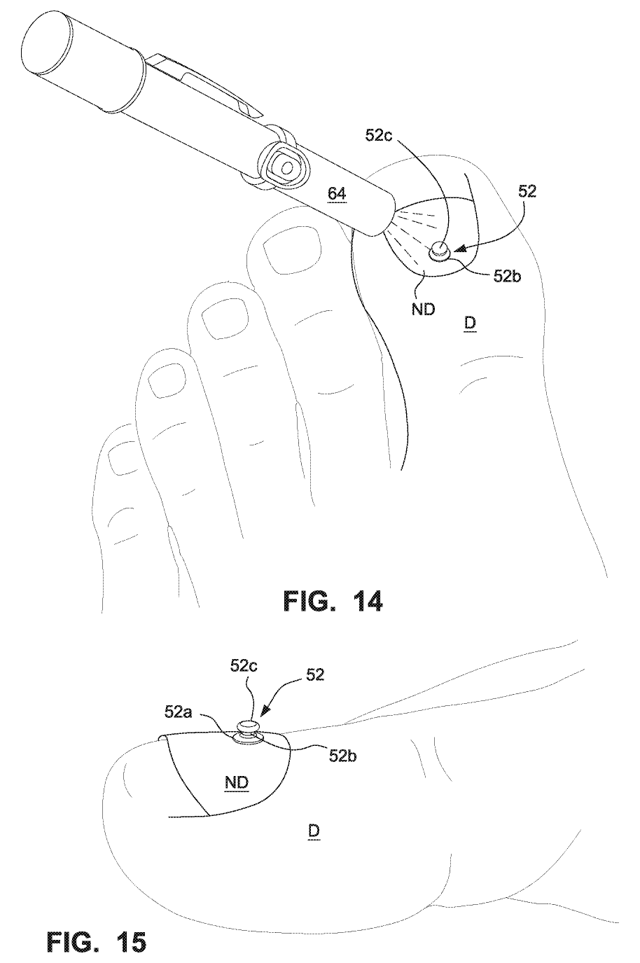

In use, the nail ND may be prepared for adhesively affixing the anchor member 52 by buffing the surface of the nail ND with the provided buffing pad 66 as shown in FIG. 11. Thereafter, a small but adequate amount of the liquid UV-curable adhesive 60a from the supply 60 thereof may be applied onto the surface of the nail at a desired location where the anchor member 52 is to be affixed. (See FIG. 12.) The anchor member 52 may then be brought into contact with the applied liquid UV-curable adhesive 60a, e.g., while gripped in the tweezers 62 to assist in the positioning and manipulation of the anchor member 52. (See FIG. 13) The UV-light emitting pen 64 may be activated when the anchor member 52 is properly positioned on the nail ND so as to expose the UV-curable adhesive to UV-light and thereby cure the same. (See FIG. 14) Once the UV-curable adhesive is cured, the anchor member 52 is immovably affixed to the nail ND as shown in FIG. 15.

Any commercially available UV-curable resin may be employed in the practice of the embodiments disclosed herein. It is presently preferred that the UV-curable resin be relatively fast curing (e.g., about 5 seconds to fully cure after exposure to UV-light) and may be clear or colored depending on patient or physician preference. One commercial UV-curable resin that may be employed satisfactorily is available from Let’s® Resin at www.letsresin.com.

The anchor member 52 is most preferably a post formed of stainless steel. Virtually any conventional stainless steel posts employed traditionally as direct bond dental appliance anchors for orthodontic purposes may be employed in the practice of this invention. One preferred stainless steel orthodontic post is a DB Button Circular Base post that may be obtained commercially from American Orthodontics of Sheboygan, Wisconsin USA. As is schematically depicted in greater detail in FIG. 10A, the anchor member 52 includes a generally circular base 52a, a central pedestal column 52b which terminates in an enlarged circular head 52c. The base 52a will thereby be adhesively affixed to the nail ND so that the central pedestal column 52b extends upwardly from the nail ND and terminates with the head 52c being spaced above the nail ND.

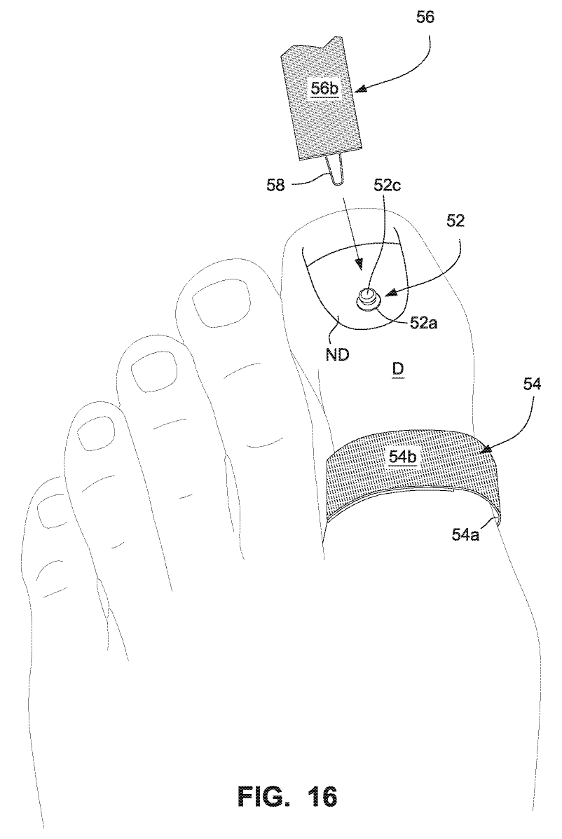

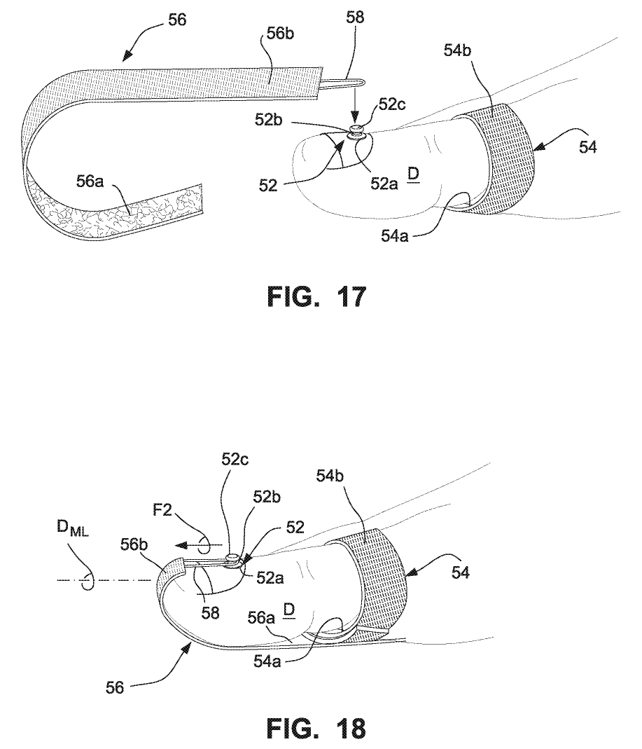

As is shown in FIGS. 16 and 17, the anchor strip 54 will be positioned about the patient’s digit D proximally of the nail ND in a similar manner as was described previously with the kit 10, i.e., with the relatively soft loop side 54a being against the patient’s skin and the hook side 54b being exposed. Once the anchor member 52 is immovably affixed to the nail ND, the traction strip 56 may be manually positioned with the loop side 56a being positioned against the patient’s digit D such that the flexible thread or wire loop 58 is extended around the head 52c of the anchor member 52 and engaged with the central pedestal column 52b thereof. The opposite tail end of the traction strip 56 may thus be extended around the distal end of the patient’s digit D and then proximally extended under the digit D such that a tail end portion of the loop side 56a of the traction strip 56 is engaged with a corresponding subjacent portion of the hook side 54b of the anchor strip 54. As was explained previously, such an engagement will exert a traction force in the distal direction (arrow F₂ in FIG. 18) thereby treat and/or enhance nail growth.

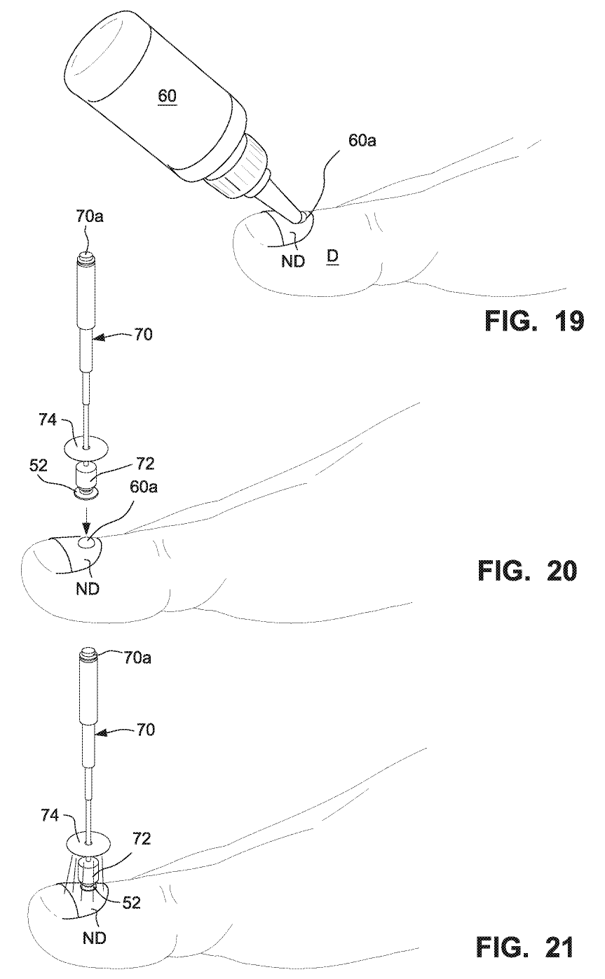

As depicted in FIGS. 19-21, a further option that may be included in the kit 50 is a telescopically extending applicator wand 70 that is provided with a magnetic attachment member 72 at a distal end thereof and a UV-light emitting collar 74 proximally spaced from the magnetic attachment member 72. As is shown in FIG. 19, an amount of UV-curable resin 60a from the supply 60 may be applied onto the nail ND as was discussed previously. The anchor member 52 (which in this embodiment must be formed of a magnetic 400 series ferritic or martensitic grade of stainless steel) may be magnetically attached to the magnetic attachment member 72 and then advanced toward the UV-curable resin 60a so as to bring the base 52a of the anchor member into contact with the applied UV-curable resin 60a.

While maintaining such positioning of the anchor member 52 by means of the attachment wand 70, the UV-light emitting collar 74 may be activated (e.g., by depressing the light activation button 70a) so as to expose the UV-curable resin 60a to UV light thereby curing the same. After the UV resin 60a is cured, the anchor member 52 will be affixed to the nail ND to thereby allow the wand 70 to be magnetically separated therefrom. Following such affixation of the anchor member 52 to the nail ND, the procedures as discussed previously with reference to FIGS. 16-18 may be performed. As can be appreciated, the attachment wand 70 will efficiently enable the patient or physician to position and bond the anchor member 52 to the nail ND using only one hand.

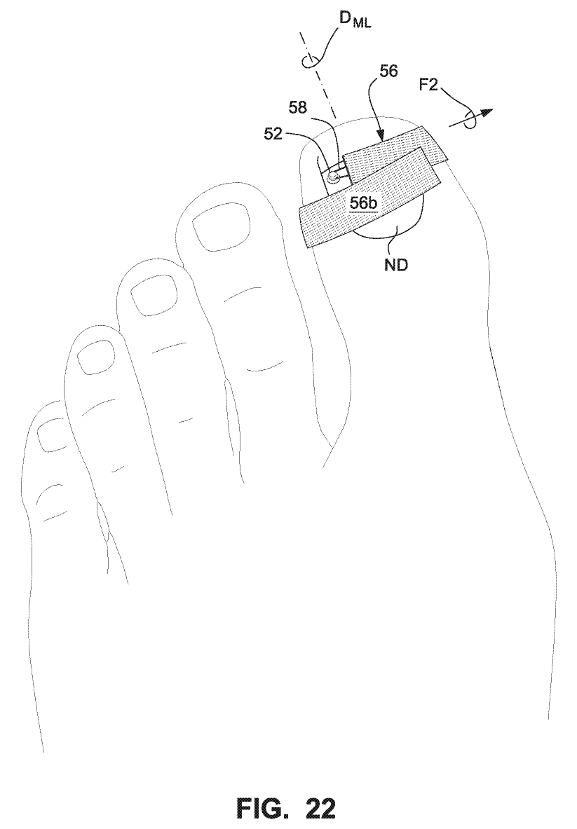

The kits 10 and 50 have been described hereinabove in a manner which promotes symmetrical growth of the nail ND in a direction aligned with the midline D_{ML} of the digit D (e.g., in the direction of the tension force F2 as shown in FIG. 18). As noted previously, however, the kits 10 and 50 could in use be employed to promote asymmetrical (e.g., substantially transverse) growth relative to the digit midline D_{ML}. By way of example, accompanying FIG. 22 schematically depicts certain of the components of the kit 50 described above that may be employed for such a purpose. As shown, the anchor member 52 may be affixed to the nail ND so as to be laterally offset relative to the midline D_{ML} of the digit D. Thus, the anchor member 52 may be affixed to the nail ND at a location that is laterally spaced from the digit midline D_{ML} but located near an area of nail ingrowth along a lateral cuticle edge of the nail ND.

The loop 58 of the elongate traction strip 56 may therefore be attached to such asymmetrically affixed anchor member 52 and then wrapped around the sides of the digit D so the fibrous loops on the surface 56a of the tail portion of the traction strip 56 opposite to the loop 58 may be brought into overlapping contact with a corresponding section of itself, i.e., with the exposed hooks on the surface 56b near the loop 58. Thus, a laterally (e.g., substantially transversely) oriented tension force (arrow F₃) relative to the digit midline D_{ML} may be exerted on the affixed anchor member 52 which will thereby assist in ameliorating nail ingrowth at the lateral cuticle edge of the nail ND. It will of course be appreciated that the anchor strip 54 will not be required to be used in the treatment technique shown in FIG. 22, but it could nonetheless be retained by the user for future possible use in the manner described above. It will also be appreciated that the components of the kit 10 as described previously could be employed in a similar manner.

### Example

A patient who had two slow growing nails (left and right great toenails) applied the device as shown and described above to only the slow growing left great toenail to determine if the device affected growth of that nail and whether there was a difference in growth with the other slow growing right great toenail, that was thereby used as a “control”. After a 3 month period of time, the before and after pictures of the patient’s left and right great toenails showed a 50-60% increase in nail growth of the left great toenail to which the device was applied as compared to the control right great toenail.

While reference is made herein to particular embodiments of the invention, various modifications within the skill of those in the art may be envisioned. Therefore, it is to be understood that the invention is not to be limited to the disclosed embodiment, but on the contrary, is intended to cover various modifications and equivalent arrangements included within the spirit and scope thereof.

What is claimed is:

1. A device kit to assist in treatment and/or growth enhancement of a nail of a patient digit in need thereof, the device kit comprising:
an anchor member adapted to being affixed to a subjacent portion of the nail of the patient digit; and
a flexible traction strip having one end connectable to the anchor member and being of sufficient length so as to extend around the digit and be attached at an opposite end thereof to thereby exert a tension force onto the anchor member in a selected direction relative to a midline of the nail of the patient digit to assist in treatment and/or enhancement of nail growth.

2. The device kit according to claim 1, which further comprises:
a flexible anchor strip having a sufficient length to be wrapped around the patient digit proximal to the nail, wherein
the flexible traction strip has a sufficient length so as to extend around a distal end of the digit and then be redirected proximally under the patient digit to allow an opposite end thereof to be attached to the anchor strip, wherein
the traction strip exerts a tension force to the nail of the patient digit substantially along the midline of the nail of the digit.

3. The device kit according to claim 2, further comprising an adhesive material which is adapted to adhesively affix the anchor member to the nail of the patient.

4. The device kit according to claim 1, wherein the traction strip is a hook-and-loop fastener strip having hooks on one surface thereof and loops on an opposite surface thereof.

5. The device kit according to claim 2, wherein at least one of the anchor strip and the traction strip is a hook-and-loop fastener strip having hooks on one surface thereof and loops on an opposite surface thereof.

6. The device kit according to claim 2, wherein each of the anchor strip and the traction strip is a flexible hook-and-loop fastener strip having hooks on one surface thereof and loops on an opposite surface thereof.

7. The device kit according to claim 1, wherein the anchor member includes a base element adapted to being affixed to the nail of the patient and a J-shaped anchor element which is oriented in a proximal direction when the anchor member is affixed to the nail of the patient.

8. The device kit according to claim 1, wherein the anchor member is a substantially flat tab having one end adapted to being affixed to the nail of the patient and an opposite unaffixed free end.

9. The device kit according to claim 8, wherein the opposite unaffixed free end of the anchor member defines an opening adapted to being connected to the traction strip.

10. The device kit according to claim 8, wherein the one end of the anchor member includes an adhesive region which is adapted to adhesively affix the one end of the anchor member to a subjacent portion of the nail of the patient.

11. The device according to claim 10, wherein the anchor member further includes a removable protective release film covering the adhesive region.

12. The device kit according to claim 1, wherein the anchor member is a metal post.

13. The device kit according to claim 12, wherein the metal post includes a generally circular base adapted to be directly bonded to the nail, a central pedestal column extending outwardly from the base and an enlarged head at terminal end of the central pedestal column which is spaced from the base.

14. The device kit according to claim 13, wherein one end of the traction strip includes a flexible thread or wire loop extending outwardly therefrom which is sized and adapted to being positioned over the enlarged head and around the central pedestal of the metal post.

15. The device kit according to claim 14, wherein the traction strip is a flexible hook-and-loop fastener strip having hooks on one surface thereof and loops on an opposite surface thereof.

16. The device kit according to claim 12, further comprising an adhesive material which adhesively attaches the anchor member to the nail.

17. The device kit according to claim 16, wherein the adhesive material is a UV-curable adhesive.

18. The device kit according to claim 17, further comprising a UV-light emitting device for curing the UV-curable adhesive when applied to the nail and thereby adhesively affix the anchor member to the nail when cured.

19. The device kit according to claim 18, wherein the UV-light emitting device comprises a UV-light pen.

20. The device kit according to claim 12, wherein the anchor member is formed of a magnetic metal, and wherein the device kit further comprises an attachment wand having a magnetic attachment member at a distal end thereof which is magnetically attachable to the anchor member.

21. The device kit according to claim 20, wherein the attachment wand comprises a UV-light emitting element proximal to the magnetic attachment member.

22. A method to treat and/or enhance nail growth of a patient in need thereof, the method comprising:
(i) providing the device kit according to claim 1;
(ii) affixing the anchor member to a subjacent portion of the nail of the patient;
(iii) connecting the one end of the traction strip to the anchor member and wrapping the traction strip around the patient’s digit; and
(iv) attaching an opposite end of the traction strip so that the traction strip exerts a tension force on the nail of the digit through the anchor member affixed thereto in a predetermined direction relative to the midline of the digit of the patient.

23. The method according to claim 22, wherein the anchor member is affixed to the nail of the patient at a location which is laterally offset relative to the midline of the digit, and wherein steps (iii) and (iv) comprising wrapping the traction strip around the digit of the patient so that the opposite end of the traction strip is brought into engagement with and is attached to the one end thereof to thereby exert a tenson force to the nail in a lateral direction relative to the midline of the nail.

24. The method according to claim 22, wherein
the device kit further comprises a flexible anchor strip having a sufficient length to be wrapped around the patient digit proximally of the nail bed, and wherein
the steps (iii) and (iv) comprise extending the flexible traction strip around a distal end of the digit and then redirecting the flexible traction strip proximally under the patient digit and attaching an opposite end thereof to the anchor strip to thereby cause the traction strip to exert a tension force to the nail of the patient digit that is substantially aligned with the midline of the nail of the digit.

25. The method according to claim 22, wherein the traction strip is a flexible hook-and-loop fastener strip having hooks on one surface thereof and loops on an opposite surface thereof.

26. The method according to claim 23, wherein each of the anchor strip and the traction strip is a flexible hook-and-loop fastener strip having hooks on one surface thereof and loops on an opposite surface thereof

27. The method according to claim 22, wherein step (ii) includes affixing the anchor member to the nail bed with adhesive.

28. The method according to claim 27, wherein step (ii) comprises (iia) applying a liquid adhesive to a portion of the nail bed, (iib) bringing the anchor member into contact with the applied liquid adhesive and (iic) allowing the adhesive to solidify.

29. The method according to claim 28, wherein the adhesive is a UV-curable adhesive, and wherein step (ii) comprises exposing the liquid adhesive to UV-light after the anchor member is in contact with the applied liquid adhesive to cure the adhesive and affix the anchor member to the nail.

30. The method according to claim 29, wherein step (ii) includes directing UV-light toward the adhesive by a UV-light emitting device.

31. The method according to claim 29, wherein the anchor member is formed of a magnetic metal, and wherein step (ii) comprises magnetically attaching the anchor member to an attachment wand having a magnetic attachment member at a distal end thereof, and manipulating the attachment wand with the anchor member attached to the magnetic attachment member so as to bring the anchor member into contact with the applied adhesive on the nail.

32. The method according to claim 31, wherein the attachment wand includes a UV-light emitting element proximal to the magnetic attachment member, and wherein following bringing the anchor member into contact with the applied liquid adhesive on the nail, step (ii) includes activating the UV-light emitting element of the attachment wand to direct UV-light toward the adhesive to cure the adhesive and affix the anchor member to the nail, followed by separating the magnetic attachment member from the anchor member.

33. The method according to claim 32, wherein the UV-light emitting element of the attachment wand includes a UV-light emitting collar positioned proximal to the magnetic attachment head.
